(19) United States
(12) Patent Application Publication
(10) Pub. No.: US 2006/0269979 A1
(43) Pub. Date: Nov. 30, 2006

(54) High throughput glycan analysis for diagnosing and monitoring rheumatoid arthritis and other autoimmune diseases

(76) Inventors: Raymond A. Dwek, Oxford (GB); Louise Royle, Oxon (GB); Pauline M. Rudd, Abingdon (GB)

Correspondence Address: FOLEY AND LARDNER LLP;SUITE 500, 3000 K STREET NW, WASHINGTON, DC, 20007, US

(21) Appl. No.: 11/411,231

(22) Filed: Apr. 26, 2006

Related U.S. Application Data
(60) Provisional application No. 60/674,724, filed on Apr. 26, 2005.
(60) Provisional application No. 60/674,722, filed on Apr. 26, 2005.

(30) Foreign Application Priority Data
Jun. 24, 2005 (WO) PCT/IB05/02995
Jun. 24, 2005 (WO) PCT/IB05/02885

(51) Int. Cl. C12Q 1/37; G01N 33/00

(52) U.S. Cl. 435/23; 436/86

(57) ABSTRACT

One can identify and quantify one or more glycosylation markers of an autoimmune disease such as rheumatoid arthritis by utilizing quantitative HPLC analysis of glycans which have been released from unpurified glycoproteins. The unpurified glycoproteins can be total glycoproteins or a selection of the total glycoproteins. The identified glycosylation marker can be utilized for monitoring and/or diagnosing the autoimmune disease.

### PRIORITY CLAIMS

This application claims priority to U.S. provisional patent applications No. 60/674,722 to Dwek et. al. filed Apr. 26, 2005, and 60/674,724 to Dwek et. al. filed Apr. 26, 2005, which are both incorporated by reference. The present application also claims priority PCT applications No. PCT/IB2005/002885 to Dwek et. al. filed Jun. 24, 2005 and PCT/IB2005/002995 to Dwek et. al. filed Jun. 24, 2005, which are both incorporated herein by reference in their entirety.

### FIELD

This invention generally relates to diagnostic and monitoring methods for rheumatoid arthritis and other autoimmune diseases and, in particular, to diagnostic and monitoring methods for rheumatoid arthritis (RA) and other autoimmune diseases based on detailed glycosylation analysis.

### BACKGROUND

RA is generally considered a systemic inflammatory disease in which an immune response by the adaptive immune system translates into an attack on the diarthrodial joints (synovium, cartilage, and bone with attendant joint destruction) and less frequently on other anatomic sites. There exists substantial evidence implicating the adaptive immune system—lymphocytes—in RA pathogenesis. Histologically, T-cells account for a portion of the mononuclear infiltrate in the synovial sublining, see Van Boxel, J. A., and S. A. Paget. Predominantly T-cell infiltrate in rheumatoid synovial membranes. New England Journal of Medicine 293:517, 1975. Genetically, the strong HLA-DR association localizing to small regions of the DRB1 *0401 and *0404 alleles (Wordsworth, B. P., et. al. HLA-DR4 subtype frequencies in rheumatoid arthritis indicate that DRB1 is the major susceptibility locus within the HLA class II region. Proceedings of the National Academy of Sciences of the United States of America 86:10049, 1989; and Ronningen, K. S., et. al. Rheumatoid arthritis may be primarily associated with HLA-DR4 molecules sharing a particular sequence at residues 67-74. Tissue Antigens 36:235, 1990) implies involvement of CD4+ T lymphocytes. There is also experimental evidence implicating B-lymphocyte and IgG involvement in RA pathogenesis. A growing list of autoantibodies associated with RA (reviewed in van Boekel, M. A., et. al. Autoantibody systems in rheumatoid arthritis: specificity, sensitivity and diagnostic value. Arthritis Res 4:87, 2002.) including serologic reactivity to keratin (anti-keratin antibodies (AKA)) (Young, B. J. et. al. “Anti-keratin antibodies in rheumatoid arthritis”, Br Med J 2:97, 1997), Sa (Despres, N. et. al. “The Sa system: a novel antigen-antibody system specific for rheumatoid arthritis”, J Rheumatol 21:1027, 1994), BiP (Blass, S., Novel 68 kDa autoantigen detected by rheumatoid arthritis specific antibodies. Ann Rheum Dis 54:355, 1995), RA33 (Hassfeld, W., G. Steiner, K. Hartmuth, G. Kolarz, O. Scherak, W. Graninger, N. Thumb, and J. S. Smolen. Demonstration of a new antinuclear antibody (anti-RA33) that is highly specific for rheumatoid arthritis. Arthritis Rheum 32:1515, 1989), glucose-6-phosphate isomerase (GPI) (Schaller, M., et. al. Autoantibodies to GPI in rheumatoid arthritis: linkage between an animal model and human disease. Nat Immunol 2:746, 2001; Kassahn, D., C. et. al. Few human autoimmune sera detect GPI. Nat Immunol 3:411, 2002; Schubert, D. et. al. Autoantibodies to GPI and creatine kinase in RA. Nat Immunol 3:411; discussion 412, 2002) and anti-perinuclear factor (APF or anti-fillagrin) (Nienhuis, L. F., and E. A. Mandema. A new serum factor in patients with rheumatoid arthritis. The antiperinuclear factor. Annals of Rheumatic Disease 23:302, 1964). Additionally, the frequent presence of rheumatoid factor in patients with RA and the recent demonstration that B-lymphocyte ablative therapy is an effective RA therapeutic (Edwards, J. C., et. al. Efficacy of B-cell-targeted therapy with rituximab in patients with rheumatoid arthritis. N Engl J Med 350:2572, 2004) points to dysregulation of the humoral adaptive immune response in these patients. Furthermore, as in the case for T-lymphocytes, B-cells are frequently found in the synovial mononuclear infiltrate in RA. With discrete differences, these lymphocytes can organize into aggregates similar to those found in lymph nodes and Peyer's patches (Rooney, M., A. et. al. The immunohistologic features of synovitis, disease activity and in vitro IgM rheumatoid factor synthesis by blood mononuclear cells in rheumatoid arthritis. Journal of Rheumatology 16:459, 1989). Taken together, these findings implicate autoimmunity involving T-lymphocytes, B-lymphocytes and IgG in the pathogenesis of RA. A clear correlation between RA and the percentage of the galactosylation on N-glycans released from purified immunoglobulin G (IgG) has been established in Parekh et al., see “Association of Rheumatoid Arthritis and Primary Osteoarthritis with Changes in the Glycosylation Pattern of Total Serum IgG,” Nature, 316, pp. 452-457, 1985, incorporated herein by reference in its entirety. In addition, the specific activity of galactosyltransferase towards asialo-agalacto IgG was found to be reduced to 50-60% of control levels in adult RA, see Parekh et. al. “Galactosylation of IgG Associated Oligosaccharides Is Reduced in Patients with Adult and Juvenile Onset Rheumatoid Arthritis and Is Related to Disease Activity”, Lancet, No. 8592, vol. 1, pp. 966-969, 1988, incorporated herein by reference in its entirety. Various glycosylation changes were also identified for other autoimmune diseases. For example, IgG glycosylation profiling distinguishes between a range of rheumatic diseases, see Watson, M., Rudd, P. M., Bland, M., Dwek, R. A. and Axford, J. S, Sugar Printing Rheumatic Diseases. A Potential Method for Disease Differentiation Using Immunoglobulin G Oligosaccharides. Arthritis and Rheumatism, vol. 42(8), pp. 1682-1690, 1999, incorporated herein by reference in its entirety.

The relationship established between rheumatoid arthritis and the galactosylation on N-glycans from purified IgG led to a so-called ‘classic’ diagnostic method for rheumatoid arthritis, see Parekh, et. al. Nature, 316, pp. 452-457, 1985. The ‘classic’ diagnostic method is described also, for example, in U.S. Pat. No. 4,659,659 “Diagnostic Method for Diseases Having an Arthritic Component” to Dwek et. al. issued on Apr. 21, 1987, incorporated herein by reference in its entirety. In the ‘classic’ diagnostic method, analyzed glycans are released from purified glycoproteins, e.g. immunoglobulin G (IgG) of serum or other body fluid. Methods for diagnosing and monitoring diseases based on mass-spectrometric measuring of glycosylation profiles of glycans released from purified glycoproteins are also disclosed in US patent application publication “Glycan Markers for Diagnosing and Monitoring Disease” No. 2004/0147033 to Shriver et. al. published on Jul. 29, 2004. Sample preparations in the classic diagnostic method for RA and methods of US patent application publication No. 2004/0147033 require purifying glycoproteins. This step can be lengthy in time and can require large amounts of serum or other body fluid, thus, making the “classical” method incompatible with a high throughput diagnostics and monitoring methods. Overcoming this problem, Butler et. al. demonstrated that a glycosylation analysis can be performed on glycans released directly from whole serum glycoproteins without glycoprotein purification, see Butler, M., Quelhas, D., Critchley, A. J., Carchon, H., Hebestreit, H. F., Hibbert, R. G., Vilarinho, L., Teles, E., Matthijs, G., Schollen, E., Argibay, P., Harvey, D. J., Dwek, R. A., Jaeken, J. and Rudd, P. M. (2003). “Detailed glycan analysis of serum glycoproteins of patients with congenital disorders of glycosylation indicates the specific defective glycan processing step and provides an insight into pathogenesis.” Glycobiology 13: 601-22, incorporated herein by reference in its entirety. Although Butler et. al. eliminated the step of glycoprotein purification, the glycan profiles and analysis were flawed because hydrazinolysis was used to release the glycans. Using hydrazinolysis for glycan release results in the desialylation of the significant proportion of the glycans and the introduction of a number of artifacts such as a loss of N-acetyl and N-glycolyl groups from the amino sugar residues (which can be subsequently re N-acetylated and this can result in both under and over acetylation), as well as loss of O-acetyl substitutions in sialic acids. Callewaert et. al. used capillary electrophoresis for analysis of glycans released from a total serum of patients, see Callewaert et. al. Electrophoresis, 2004, 25, 3128-3131. However, the Callewaert et. al. were able to identify only the major desialylated structures. Thus, it is highly desirable to develop a method of diagnosing and monitoring of rheumatoid arthritis and other autoimmune diseases based on a detailed glycosylation analysis of glycans of glycoproteins released from a body fluid or a body tissue which would not require glycoprotein purification and the use of hydrazinolysis for the release of glycans.

### SUMMARY

According to one embodiment, one can identify and/or quantify one or more glycosylation markers of an autoimmune disease by a method comprising (A) obtaining a sample from a subject diagnosed with the autoimmune disease; (B) relasing glycans of unpurified glycoproteins of the sample; (C) measuring a glycosylation profile of the glycans by quantitative high performance liquid chromatography (HPLC) alone or in combination with mass spectrometry and (D) comparing the glycosylation profile with a control profile to determine the one or more glycosylation markers of the autoimmune disease.

According to another embodiment, one can diagnose, monitor and/or prognosticate an autoimmune disease in a subject by a method comprising (A) obtaining a sample from the subject; (B) releasing glycans of unpurified glycoproteins of the sample; (C) measuring a glycosylation profile of the glycans by quantitative HPLC alone or in combination with mass spectrometry; and (D) comparing the glycosylation profile with a control profile to determine a level of one or more glycosylation markers of the disease.

According to yet another embodiment, one can diagnose, monitor and/or prognosticate rheumatoid arthritis in a subject by a method comprising (A) obtaining a sample from the subject; (B) releasing glycans of glycoproteins of the sample; and (C) measuring a glycoprofile of the glycans to determine a ratio between an amount of G0 glycans and an amount of G1 glycans.

### BRIEF DESCRIPTION OF THE DRAWINGS

FIG. 1 shows sodium dodecyl sulphate polyacryl amide gel electrophoresis (SDS-PAGE) and normal phase high performance liquid chromatography (NP-HPLC) profiles of glycans released from purified immunoglobulin G (IgG) of samples GBRA13 and GBRA1.

FIG. 2 shows NP-HPLC profiles of glycans released from purified IgG of sample GBRA15.

FIG. 3 shows NP-HPLC profiles of control and sample GBRA15.

FIG. 4 shows a correlation between G0/tripleG1 versus G0 as a percentage of total purified IgG glycans for purified IgG glycans.

FIG. 5 shows a correlation between G0/tripleG1 from serum versus purified IgG.

FIG. 6 shows a correlation between G0/tripleG1 for glycans released from whole serum and G0 as a percentage of total glycans released from purified IgGs.

FIG. 7 shows G0/tripleG1 ratios in glycans released from whole serum using polyvinyldene fluoride (PVDF) membranes (serum PVDF) and in glycans released from purified IgG heavy chain gel bands (purified IgG heavy chain gel band).

### DETAILED DESCRIPTION

Unless otherwise indicated, “a” or “an” means “one or more”.

The present invention relates to diagnostic and monitoring methods for autoimmune diseases and, in particular, to diagnostic and monitoring methods for autoimmune diseases based on detailed glycosylation analysis of glycans of glycoproteins.

This application incorporates by reference in their entirety U.S. provisional patent application No. 60/674,724 “An automated glycofingerprinting strategy” to Dwek et. al. filed Apr. 26, 2005, and U.S. provisional patent application No. 60/674,723 “Glycosylation markers for cancer diagnostics and monitoring” to Dwek et. al. filed Apr. 26, 2005.

Unless otherwise specified, “a” or “an” means “one or more”.

“Glycoprotein” designates an amino acid sequence and one or more oligosaccharide (glycan) structures associated with the amino acid sequence.

“Glycoprofile” or “glycosylation profile” means a presentation of glycan structures (oligosaccharides) present in a pool of glycans . A glycoprofile can be presented, for example, as a plurality of peaks each corresponding to one or more glycan structures present in a pool of glycans.

“Glycosylation marker” means a particular difference in glycosylation between a sample of a subject diagnosed with an autoimmune disease and a sample from healthy control.

“Control profile” means a glycosylation profile from a sample not affected by the autoimmune disease. The control sample can originate from a single individual or be a sample pooled from more than one individuals.

The term “subject” means an animal, more preferably a mammal, and most preferably a human.

The high throughput format can mean a standard multiwell format such as 48 well plate or 96 well plate.

The term “autoimmune disease” includes but is not limited to the following diseases: such as rheumatoid arthritis, osteoarthritis, juvenile chronic arthritis, systematic lupus erythematosus, Sjogren's syndrome, ankylosing spondylitis, psoriatic arthritis, multiple sclerosis, inflammatory bowel disease, graft-vs-host disease and scleroderma.

The present application incorporates by the reference in their entirety US application “Automated Strategy for Identifying Physiological Glycosylation Marker(s)” to Dwek et. al. filed Apr. 26, 2006, and US application “Glycosylation Markers for Cancer Diagnosing and Monitoring” to Dwek et. al. filed Apr. 26, 2006.

The inventors have recognized that one can identify and/or quantify one or more glycosylation markers of an autoimmune disease by measuring a detailed glycosylation profile of glycans that have been released from,unpurified glycoproteins of a sample from a subject diagnosed with the autoimmune disease. The advantages of using the unpurified glycoproteins, i.e. omitting a step of glycoprotein purification, in the glycosylation analysis can be a reduced time required for a sample preparation and a reduced amount of a sample material used. Also the use of the unpurified glycans makes the present methodology compatible with a high through format such as a multiwell plate format.

The sample can be any sample that contains glycoproteins. The sample can be, for example, a sample of a body tissue or a sample of a body fluid such as whole serum, blood plasma, synovial fluid, urine, seminal fluid or saliva.

The unpurified glycoproteins can be total glycoproteins in the sample, i.e. all the glycoproteins in the sample without any loss. The unpurified glycoproteins can also be a selection of total glycoproteins in the sample. Such selection is not limited to a single type of glycoprotein but still represents a pool or plurality of different types of glycoproteins, i.e. to glycoproteins having different amino acid sequences.

Preferably, glycans are released in such a way so that they are not modified, i.e. the released glycans are the native glycans of the glycoproteins of the sample. In some embodiments, glycans can be released from unpurified glycoproteins in solution. Yet in some embodiments, glycans can be released from immobilized unpurified glycoproteins. In some embodiments, unpurified glycoproteins can be immobilized in a high throughput format such as a multiwell plate.

In some embodiments, the unpurified glycoproteins can be total glycoproteins from the sample that are immobilized in a non-selective format such as gel block. Yet in some embodiments, the unpurified glycoproteins can be a selection of total glycoproteins immobilized on a protein binding membrane such as a PVDF membrane or in a gel piece such as a gel band or a gel spot.

The measurement of the glycoprofile of the released glycans can be carried out by quantitative HPLC alone or in combination with mass spectrometry. The measured glycoprofile can then be compared with a control glycoprofile to determine one or more glycosylation markers of the autoimmune disease. Comparing the glycoprofiles can involve comparing peak ratios in the profiles. When more than one glycosylation marker is identified, one can select one or more of the markers that have the highest correlation with one or more parameters of the subject diagnosed with the autoimmune disease. Such parameters can be diagnosis, age, sex, disease stage, disease activity, disease prognosis, remission, response to a therapy, medical history or any combination thereof.

The identified glycosylation marker of an autoimmune disease can be used for diagnosing, monitoring and/or prognosticating an autoimmune disease in a subject by measuring a glycoprofile of glycans that have been released from glycoproteins from a sample of the subject to determine a level of the glycosylation marker in the subject.

Measuring of the glycoprofile and determining the level of the identified glycosylation marker can be carried out by any suitable, i.e. not necessarily by the technique used to identified the glycosylation marker initially. Examples of such alternative techniques can be capillary electrophoresis and lectin chromatography.

For determining a level of the identified glycosylation marker, one can measure a glycosylation profile of glycans that have been released from either unpurified glycoproteins or from purified, i.e. isolated glycoproteins, such as serum immunoglobulin G (IgG), serum immunoglobulin A (IgA), IgM, complement components or inflammatory markers.

The identified glycosylation marker can be used for an effect of therapy against an autoimmune disease by comparing levels of the glycosylation marker before and after treatment of a subject with the therapy. One can also use the identified glycosylation marker for adjusting and/or optimizing a dose of a therapeutic agent or for testing a new therapy or a new therapeutic agent for treating the autoimmune disease.

One example of the glycosylation marker identified according to the methodology of the present invention can be a glycosylation marker for rheumatoid arthritis which is a ratio between an amount of G0 glycans, i.e. glycans having no galactose, and an amount of G1 glycans, i.e. glycans having exactly one galactose, in a measured glycosylation profile.

Releasing Glycans

Glycans can be released from a sample of a subject such as a sample of a body fluid or a body tissue. The sample of the body fluid can be, for example, a sample of whole serum, blood plasma, urine, seminal fluid, seminal plasma, feces or saliva. The released glycans can be N-glycans or O-glycans.

In some embodiments, releasing a glycan pool of glycoproteins from a sample of a sample can be carried out without purifying the glycoproteins. In other words, the released glycans are glycans of all or substantially all of the glycoproteins present in the sample rather than of one or more purified and isolated glycoproteins.

In some embodiments, substantially all of the glycoproteins can mean all the glycoproteins that are recovered, yet in some embodiments substantially all of the glycoproteins can mean all the glycoproteins except those that are specifically removed. Releasing glycans can be carried out without exposing the sample to hydrazinolysis. In some embodiments, releasing glycans can be carried out from a very small sample of a body fluid. In some embodiments, samples of a body fluid can be less than 100 microliters, yet preferably less than 50 microliters, yet more preferably less than 20 microliters, yet more preferably less than 10 microliters, yet most preferably less than 5 microliters. The present methods of releasing can be optimized to work with body fluid samples of less than 1 microliters.

In some embodiments, releasing glycans can comprise releasing glycans from total glycoproteins the sample in solution. Yet in some embodiments, releasing glycans can comprise immobilizing total glycoproteins of the sample, for example, on protein binding membrane or in a gel. The protein binding membrane can be any protein binding membrane, for example, polyvinyldene fluoride (PVDF) membrane, nylon membrane or Polytetrafluoroethylene (PTFE) membrane. In some embodiments, releasing glycans can further comprise releasing glycans from the total glycoproteins immobilized on the protein binding membrane or in the gel. When released glycans are N-linked glycans, releasing glycans from the immobilized glycoproteins can be carried out using enzymatic release with, for example, peptide N glycosidase F.

When the glycoproteins are immobilized in the gel, releasing glycans can comprise separating the gel into a plurality of bands and selecting one or more bands from the plurality of bands from which the glycans are subsequently released (in gel band method). In some embodiments, releasing glycans from the gel can be carried out from the total gel, i.e. without separating gel into the bands. In some embodiments, releasing glycans is carried out by chemical release methods, such as β-elimination or ammonia-based β-elimination, which can be used for releasing N-linked or O-linked glycans from glycoproteins in solution or from glycoproteins immobilized on protein binding membrane. For using the methods of this invention in a high throughput format, it may be preferred to release a glycan pool from total glycoproteins immobilized in a gel or on a protein binding membrane as it can allow to use smaller samples of body fluid or body tissue.

The details of some of the release methods and their applicability to both N-glycans and O-glycans are discussed below, however, it should be understood that the present invention is not limited to the discussed below release methods.

In-gel-band: This method can be used for N-glycan release from single glycopeptides in sodium dodecyl sulphate polyacrylamide gel electrophoresis (SDS PAGE) gel bands and is based on the method described in Kuster, B., Wheeler, S. F., Hunter, A. P., Dwek, R. A. and Harvey, D. J. (1997) “Sequencing of N-linked oligosaccharides directly from protein gels: in-gel deglycosylation followed by matrix-assisted laser desorption/ionization mass spectrometry and normal-phase high-performance liquid chromatography.” Anal-Biochem 250: 82-101, incorporated herein by reference in its entirety. Samples can be reduced and alkylated by adding 4 μl of 5× sample buffer (5× sample buffer: 0.04 g Bromophenol blue, 0.625 ml 0.5M Tris (6 g for 100 ml) adjusted to pH 6.6 with HCl, 1 ml 10% SDS, 0.5 ml glycerol, in 2.875 ml water), 2 μl of 0.5M dithiothreitol (DTT) and water to make up to 20 μl in total, incubated at 70° C. for 10 min, then alkylated by addition of 2 μl of 100 mM iodoacetamide and incubated for 30 min in the dark at room temperature. Samples can be then separated on SDS-PAGE gels after which the proteins are stained with Coomassie brilliant blue, the band of interest is excised and destained. Subsequently, the gel band can be cut into 1 mm³ pieces and frozen for 2 hours or more (this can help break down the gel matrix). This gel band can be then washed alternatively with 1 ml of acetonitrile then 1 ml of digestion buffer (20 mM NaHCO₃ pH 7), which can be repeated twice before the gel plug can be then dried. PNGase F buffer solution (30 μl of 100 U/ml) is added (this is enough for 10-15 mm³ gel), more enzyme solution is added if larger gel bands can be used. The PNGaseF and gel pieces can be incubated overnight at 37° C. The supernatant can be recovered along with 3×200 μl water washes (with sonication with gel pieces for 30 mins each) followed by an acetonitrile wash (to squeeze out the gel), another water wash and a final acetonitrile wash. Samples can be filtered through a 0.45 μm LH Millipore filter and dried down for fluorescent labeling.

In-gel-block: To avoid the problems with clean up of samples following solution phase enzymatic glycan release an in-gel-block release from protein mixtures can be used. Briefly, the whole protein mixture (e.g. serum or plasma) can be reduced and alkylated as in the In-gel band oligosaccharide release described above, then set into 15% SDS-gel mixture but without bromophenol blue. A total volume of gel of 185 μl can be used (initially set into a 48 well plate, then removed for cutting up) with 300 μl of 100 U/ml of PNGaseF. The washing procedures can be similar to those used for in-gel-band release. Washing of gel can allow separation of the glycan pool from the parent proteins and thus provides glycans suitable for fluorescent labeling and further HPLC analysis. The in-gel-block procedure can be more suitable for automated glycan release than in-solution PNGaseF release, and can be the preferred method for high throughput glycan analysis.

This in-gel-block method has been further modified to work with smaller amounts of gel set into a 96 well plate. One can reduce and alkylate 5 μl of serum, in a polypropylene 96 well flat bottomed microplate, then set the sample into a gel-block by adding 30% (w/w) acrylamide: 0.8% (w/v) bis-acrylamide stock solution (37.5:1) (Protogel ultrapure protein and sequencing electrophoresis grade, gas stabilised; National Diagnostics, Hessle, Hull, UK), 1.5M Tris pH 8.8, 10% SDS, 10% APS (ammonium peroxodisulphate) and finally TEMED (N,N,N,N′-Tetramethyl-ethylenediamine) mixing then leave it to set. The gel blocks can be then transferred to a filter plate (Whatman protein precipitation plate) then washed with acetonitrile followed by 20 mM NaHCO₃. The gel pieces can be then dried in a vacuum centrifuge, incubated with 1% formic acid at for 40 min and then re-dried. The N-glycans can be released incubating with PNGaseF solution (Roche Diagnostics GmbH, Mannheim, Germany. The released glycans can be collected into a 2 ml square tapered polypropylene 96 well plate by washing the gel pieces with water followed by acetonitrile. The released glycans can be dried then labeled by incubating with 2-AB labelling solution (LudgerTag 2-AB labelling kit), for 2 hours at 65° C. Excess 2AB can be removed using a HILIC solid phase extraction (SPE) micro-elution plate (Waters) in a vacuum manifold. The labeled glycans can then eluted into a 2 ml 96 well then dried and redissolved them in 50 mM ammonium formate and acetonitrile ready for HPLC.

Enzymatic release of N-glycans from PVDF membranes. The glycoproteins in reduced and denatured serum samples can be attached to a hydrophobic PVDF membrane in a 96 well plate by simple filtration. The samples can be then washed to remove contaminates, incubated with PNGaseF to release the glycans based on the methods described in Papac, D. I., et. al. Glycobiology 8: 445-54, 1998, and in Callewaert, N., et. al. Electrophoresis 25: 3128-31, 2004, both incorporated herein by reference in their entirety. The N-glycans can be then washed from the bound protein, collected and dried down ready for fluorescent labeling. N-glycans can be released in situ from the glycoproteins by incubation with PNGaseF and by chemical means. The 2AB labeled N-glycans can be cleaned by SPE as in the in-gel-block method.

Chemical release of N— and O-glycans. In contrast to the advantages that enzymatic release of N-glycans can afford to N-glycan analysis, no enzymatic methodology currently exists for the release of structurally intact O-glycans. Chemical release by reductive β-elimination can require the concomitant reduction of the released oligosaccharides to their alditol derivatives (Amano, J. et. al. Methods Enzymol 179: 261-70, 1989) to prevent degradation (peeling). This reduction precludes the use of any post-release labeling so that detection is limited to mass spectrometry, pulsed amperometric detection and/or radioactivity.

Ammonia-based β-elimination can be used to release both N— and O-glycans by a modification of the classical β-elimination (Huang, Y. et. al. Analytical Chemistry 73: 6063-6069, 2001) which can be applied to glycoproteins in solution or on PVDF membranes. Ammonia-based P-elimination can be done from PVDF membranes.

This strategy, can be optimized for high throughput, and can provide a powerful approach for releasing both N— and O-glycans in their correct molar proportions and in an open ring form suitable for post-release labeling.

Release of N— and O-glycans from protein binding PVDF membranes by ammonia based beta-elimination. Samples of glycoprotein, mixtures of glycoproteins, whole serum or other body fluids can be reduced and alkylated as in the in-gel-band method. Protein binding PVDF membranes (Durapore 13 mm×0.45 μm HVHP, Millipore) in Swinnex filter holders (Millipore) can be pre-washed with 2×2.5 ml water using an all-polypropylene 2.5 ml syringe (Sigma), followed by a syringe full of air to remove most of the liquid from the membrane. The reduced and alkylated sample can be then applied directly to the membrane and left to bind for 5 min before washing by pushing through 2×2.5 ml water slowly with a syringe, followed by a syringe full of air to remove most of the liquid from the membrane. The filter with the bound glycoprotein samples can be then carefully removed from the filter holder and placed in a 1.5 ml screw capped polypropylene tube with a molded PTFE cap. 1 ml of ammonium carbonate saturated 29.2% aqueous ammonium hydroxide, plus 100 mg ammonium carbonate can be added to the tube. This can be incubated for 40 hours at 60° C., then cooled in the fridge. The liquid can be then transferred to a clean tube and evaporated to dryness. The released glycans can be re-dissolved in water and re-dried until most of the salts are removed. 100 μl of 0.5M boric acid can be added to the glycans and incubated at 37° C. for 30 min. The glycans can be then dried under vacuum, 1 ml methanol added, re-dried, a further 1 ml methanol can be added and re-dried to remove the boric acid.

Quantitatively Analyzing the Glycans.

Labeling of glycans. In some embodiments, upon releasing, the glycans can be labeled with, for example, a fluorescent label or a radioactive label. The fluorescent label can be, for example, 2-aminopyridine (2-AP), 2-aminobenzamide (2-AB), 2-aminoanthranilic acid (2-AA), 2-aminoacridone (AMAC) or 8-aminonaphthalene-1,3,6-trisulfonic acid (ANTS). Labeling of glycans with fluorescent labels is described, for example, by Bigge, J. C., et. al. “Nonselective and efficient fluorescent labeling of glycans using 2-amino benzamide and anthranilic acid.” Anal Biochem 230: 229-38, 1995, incorporated herein reference in its entirety, and Anumula, K. R. (2000). High-sensitivity and high-resolution methods for glycoprotein analysis. Analytical Biochemistry 283: 17-26, incorporated by reference in its entirety.

Fluorescent labels can label all glycans efficiently and non-selectively and can enable detection and quantification of glycans in the sub picomole range. The choice of fluorescent label depends on the separation technique used. For example, a charged label is specifically required for capillary electrophoresis. In particular, 2-AB label can be preferred for chromatographic, enzymatic and mass spectroscopic processes and analyses, while 2-AA label can be preferred for electrophoretic analyses.

Unlabelled glycans can be also detected by, for example, mass spectrometry, however, fluorescent labelling may aid glycan ionisation, see e.g. Harvey, D. J. (1999). “Matrix-assisted laser desorption/ionization mass spectrometry of carbohydrates.” Mass Spectrom Rev 18: 349-450.; Harvey, D. J. (2000). Electrospray mass spectrometry and fragmentation of N-linked carbohydrates derivatized at the reducing terminus. J Am Soc Mass Spectrom 11: 900-915.

Measuring glycoprofile of the released glycans. Glycoprofile of the glycans means a presentation of particular glycan structures in the glycans. Measuring glycoprofile of the glycans can be carried out by quantitative analytical technique, such as chromatography, mass spectrometry, electrophoresis or a combination thereof. In particular, the chromatographic technique can be high performance anion exchange chromatography (HPAEC), weak ion exchange chromatography (WAX), gel permeation chromatography (GPC), high performance liquid chromatography (HPLC), normal phase high performance liquid chromatography (NP-HPLC), reverse phase HPLC (RP-HPLC), or porous graphite carbon HPLC (PGC-HPLC). The mass spectrometry technique can be, for example, matrix assisted laser desorption/ionization time of flight mass spectrometry (MALDI-TOF-MS), electrospray ionization time of flight mass spectrometry (ESI-TOF-MS), positive or negative ion mass spectrometry or liquid chromatography mass spectrometry (LC-MS). The electrophoretic technique can be, for example, gel electrophoresis or capillary electrophoresis. The use of these quantitative analytical techniques for analyzing glycans is described, for example, in the following publications:

1) Guile, G. R., Wong, S. Y. and Dwek, R. A. (1994). “Analytical and preparative separation of anionic oligosaccharides by weak anion-exchange high-performance liquid chromatography on an inert polymer column.” Analytical Biochemistry 222: 231-5 for HPLC, incorporated herein by reference in its entirety;

2) Butler, M., Quelhas, D., Critchley, A. J., Carchon, H., Hebestreit, H. F., Hibbert, R. G., Vilarinho, L., Teles, E., Matthijs, G., Schollen, E., Argibay, P., Harvey, D. J., Dwek, R. A., Jaeken, J. and Rudd, P. M. (2003). “Detailed glycan analysis of serum glycoproteins of patients with congenital disorders of glycosylation indicates the specific defective glycan processing step and provides an insight into pathogenesis.” Glycobiology 13: 601-22, for MALDI-MS, NP-HPLC and ESI-liquid chromatography/MS, incorporated herein by reference in its entirety;

3) Jackson, P., Pluskal, M. G. and Skea, W. (1994). “The use of polyacrylamide gel electrophoresis for the analysis of acidic glycans labeled with the fluorophore 2-aminoacridone.” Electrophoresis 15: 896-902, for polyacrylamide gel electrophoresis (PAGE), incorporated herein by reference in its entirety;

4) Hardy, M. R. and Townsend, R. R. (1994). “High-pH anion-exchange chromatography of glycoprotein-derived carbohydrates.” Methods Enzymol 230: 208-25, for HPAEC using pulsed amperometric detection (PAD), incorporated herein by reference in its entirety;

5) Callewaert, N., Contreras, R., Mitnik-Gankin, L., Carey, L., Matsudaira, P. and Ehrlich, D. (2004). “Total serum protein N-glycome profiling on a capillary electrophoresis-microfluidics platform.” Electrophoresis 25: 3128-31 for capillary electrophoresis, incorporated herein by reference in its entirety;

6) Guile, G. R., Rudd, P. M., Wing, D. R., Prime, S. B. and Dwek, R. A. (1996). “A rapid high-resolution high-performance liquid chromatographic method for separating glycan mixtures and analyzing oligosaccharide profiles.” Anal Biochem 240: 210-26, for HPLC, incorporated herein by reference in its entirety;

7) Caesar, J. P., Jr., Sheeley, D. M. and Reinhold, V. N. (1990). “Femtomole oligosaccharide detection using a reducing-end derivative and chemical ionization mass spectrometry.” Anal Biochem 191: 247-52, for LC-MS, incorporated herein by reference in its entirety;

8) Mattu, T. S., Royle, L., Langridge, J., Wormald, M. R., Van den Steen, P. E., Van Damme, J., Opdenakker, G., Harvey, D. J., Dwek, R. A. and Rudd, P. M. (2000). “O-glycan analysis of natural human neutrophil gelatinase B using a combination of normal phase-HPLC and online tandem mass spectrometry: implications for the domain organization of the enzyme.” Biochemistry 39: 15695-704, for NP-HPLC and MS, incorporated herein by reference in its entirety;

9) Royle, L., Mattu, T. S., Hart, E., Langridge, J. I., Merry, A. H., Murphy, N., Harvey, D. J., Dwek, R. A. and Rudd, P. M. (2002). “An analytical and structural database provides a strategy for sequencing O-glycans from microgram quantities of glycoproteins.” Anal Biochem 304: 70-90, for NP-HPLC and MS, incorporated herein by reference in its entirety;

10) Anumula, K. R. and Du, P. (1999). “Characterization of carbohydrates using highly fluorescent 2-aminobenzoic acid tag following gel electrophoresis of glycoproteins.” Anal Biochem 275: 236-42, for gel electrophoresis, incorporated herein by reference in its entirety;

11) Huang, Y. and Mechref, Y. (2001). “Microscale nonreductive release of O-linked glycans for subsequent analysis through MALDI mass spectrometry and capillary electrophoresis.” Analytical Chemistry 73: 6063-6069, for a combination of MALDI-MS and capillary electrophoresis, incorporated herein by reference in its entirety;

12) Burlingame, A. L. (1996). “Characterization of protein glycosylation by mass spectrometry.” Curr Opin Biotechnol 7: 4-10, for mass spectrometry, incorporated herein by reference in its entirety;

13) Costello, C. E. (1999). “Bioanalytic applications of mass spectrometry.” Curr Opin Biotechnol 10: 22-8, for mass spectrometry, incorporated herein by reference in its entirety;

14) Davies, M. J. and Hounsell, E. F. (1996). “Comparison of separation modes of high-performance liquid chromatography for the analysis of glycoprotein- and proteoglycan-derived oligosaccharides.” J Chromatogr A 720: 227-33, for HPLC, incorporated herein by reference in its entirety;

15) El Rassi, Z. (1999). “Recent developments in capillary electrophoresis and capillary electrochromatography of carbohydrate species.” Electrophoresis 20: 3134-44, for capillary electrophoresis and capillary electrochromatography, incorporated herein by reference in its entirety;

16) Kuster, B., Wheeler, S. F., Hunter, A. P., Dwek, R. A. and Harvey, D. J. (1997). “Sequencing of N-linked oligosaccharides directly from protein gels: in-gel deglycosylation followed by matrix-assisted laser desorption/ionization mass spectrometry and normal-phase high-performance liquid chromatography.” Anal-Biochem 250: 82-101, for NP-HPLC and MALDI-MS, incorporated herein by reference in its entirety;

17) Reinhold, V. N., Reinhold, B. B. and Chan, S. (1996). “Carbohydrate sequence analysis by electrospray ionization-mass spectrometry.” Methods Enzymol 271: 377-402, for ESI-MS, incorporated herein by reference in its entirety;

18) Mattu, T. S., Pleass, R. J., Willis, A. C., Kilian, M., Wormald, M. R., Lellouch, A. C., Rudd, P. M., Woof, J. M. and Dwek, R. A. (1998). “The glycosylation and structure of human serum IgA1, Fab, and Fc regions and the role of N-glycosylation on Fc alpha receptor interactions.” Journal of Biological Chemistry 273: 2260-72, for WAX and NP-HPLC, incorporated herein by reference in its entirety.

19) Callewaert, N., Schollen, E., Vanhecke, A., Jaeken, J., Matthijs, G., and Contreras, R. (2003). Increased fucosylation and reduced branching of serum glycoprotein N-glycans in all known subtypes of congenital disorder of glycosylation I. Glycobiology 13: 367-375, incorporated herein by reference in its entirety;

20) Block, T. M. Comunale, M. A., Lowman, M., Steel, L. F., Romano, P. R., Fimmel, C., Tennant, B. C. London, A. A. Evans, B. S. Blumberg, R. A. Dwek, T. S. Mattu and A. S. Mehta, “Use of targeted glycoproteomics to identify serum glycoproteins that correlate with liver cancer in woodchucks and humans”. PNAS USA (2005) 102, 779-784, incorporated herein by reference in its entirety;

21) D. J. Harvey, Fragmentation of negative ions from carbohydrates: Part 1; Use of nitrate and other anionic adducts for the production of negative ion electrospray spectra from N-linked carbohydrates, J. Am. Soc. Mass Spectrom., 2005, 16, 622-630, incorporated herein by reference in its entirety;

22) D. J. Harvey, Fragmentation of negative ions from carbohydrates: Part 2, Fragmentation of high-mannose N-linked glycans, J. Am. Soc. Mass Spectrom., 2005, 16, 631-646, incorporated herein by reference in its entirety;

23) D. J. Harvey, Fragmentation of negative ions from carbohydrates: Part 3, Fragmentation of hybrid and complex N-linked glycans, J. Am. Soc. Mass Spectrom., 2005, 16, 647-659, incorporated, herein by reference in its entirety.

Although many techniques can be used for measuring glycoprofiles, in the method of determining one or more glycosylation markers of an autoimmune disease it can be preferred to measure glycoprofiles by high performance liquid chromatography (HPLC) alone or in combination with mass spectrometry. For example, measuring glycoprofiles can be performed by gel electrophoresis (see Jackson, P., Pluskal, M. G. and Skea, W. (1994). “The use of polyacrylamide gel electrophoresis for the analysis of acidic glycans labeled with the fluorophore 2-aminoacridone.” Electrophoresis 15: 896-902); HPAEC using pulsed amperometric detection (PAD) (Townsend, R. R., Hardy, M. R., Hindsgaul, O. and Lee, Y. C. (1988). “High-performance anion-exchange chromatography of oligosaccharides using pellicular resins and pulsed amperometric detection.” Anal Biochem 174: 459-70; and Hardy, M. R. and Townsend, R. R. (1994). “High-pH anion-exchange chromatography of glycoprotein-derived carbohydrates.” Methods Enzymol 230: 208-25); or capillary electrophoresis (see El Rassi, Z. (1999). “Recent developments in capillary electrophoresis and capillary electrochromatography of carbohydrate species.” Electrophoresis 20: 3134-44), however, these techniques are not ideally suited to large-scale automation, nor do they provide full quantitative structural analysis. In general they have poor detection limits, low reproducibility and are restricted by the inherent difficulty of obtaining full structural characterization of the oligosaccharides and the lack of predictability that is required to enable the preliminary assignments to be made to novel structures.

Measuring a glycoprofile by quantitative HPLC, i.e. measuring a glycoprofile of fluorescently labeled glycans such as 2AB labeled glycans by HPLC can allow accurate quantification and structural assignment of the glycan structures in the glycan pool by integration of the peaks in the chromatogram. The fluorescent labeling is non-selective and adds one fluorescent label per glycan, thus, allowing a direct correlation between fluorescence measured as peak area or height and the amount of each glycan. For an HPLC measured glycoprofile, glycan structures present in the analyzed glycan pool are separated based on their elution time. For NP-HPLC, the elution times can be converted to glucose units by comparison with a standard dextran hydrolysate ladder. An HPLC measured glycoprofile can trace all glycan structures present in a glycan pool in correct molar proportions. Polar functional groups of stationary phase of HPLC can interact with the hydroxyl groups of the glycans in a manner that is reproducible for a particular monosaccharide linked in a specific manner. For example, the contribution of the outer arm fucose addition is much greater than the addition of a core fucose residue; a core fucose residue always contributes 0.5 glucose units (gu) to the overall elution position. The characteristic incremental values associated with different monosaccharide additions can allow the preliminary assignment of a predicted structure for a particular peak present in the glycoprofile. This structure can be then confirmed by digestion with exoglycosidase arrays and/or mass spectrometry. Other techniques, such as capillary electrophoresis are not as predictable as NP-HPLC. Although, CE migration times can be calibrated with standards, the migration times of unknown structures can not be easily predicted.

Measuring glycoprofiles by NP-HPLC can be also preferred for the following reason.

Digestion of a glycan pool with one or more exoglycosidases removes monosaccharide residues and, thus, decreases the retention times or associated gu values in the glycoprofile measured by NP-HPLC. In some embodiments, this can enable the segregation of the peaks that are associated with one or glycosylation markers by shifting away peaks that are not related to the glycosylation changes away from the measured region of the glycoprofile.

In some embodiments, measuring glycoprofiles can be carried out using reverse phase high performance liquid chromatography. For RP-HPLC measured glycoprofiles, the elution times can be converted into arabinose units using a standard arabinose ladder.

The use of RP-HPLC for measuring glycosylation profiles is described, for example, in Guile, G. R., Harvey, D. J., O'Donnell, N., Powell, A. K., Hunter, A. P., Zamze, S., Fernandes, D. L., Dwek, R. A., and Wing, D. R. (1998). “Identification of highly fucosylated N-linked oligosaccharides from the human parotid gland. European Journal of Biochemistry” 258: 623-656; Royle, L., Mattu, T. S., Hart, E., Langridge, J. I., Merry, A. H., Murphy, N., Harvey, D. J., Dwek, R. A., and Rudd, P. M. (2002).

An analytical and structural database provides a strategy for sequencing O-glycans from microgram quantities of glycoproteins. Analytical Biochemistry 304: 70-90, incorporated herein by reference. RP-HPLC measured glycoprofiles can be used to complement glycoprofiles measured by NP-HPLC. For example, RP-HPLC can separate bisected glycan structures from glycan structures that do not contain bisecting N-acetylglucoamine residue. In NP-HPLC measured glycoprofiles these structures can be too close to be resolved. In some embodiments, measuring glycoprofiles by RP-HPLC can comprise using one or more buffers. The mobile phase can be used, for example, to improve the reproducibility of the measurement. The buffer can be, for example, solvent A: 50 mM of ammonium formate adjusted to pH 5 with triethylamine and solvent B: solvent A and acetonitrile mixed 50/50.

In some embodiments, HPLC can be used as a preparative method for collecting glycans, i.e. HPLC can be used to isolate unusual glycans for further analysis, by e.g. mass spectrometry, as well as for obtaining parameters for a glycan database.

In some embodiments, each of the glycoprofiles can be presented as a plurality of peaks corresponding to glycan structures in the glycans. In the method of determining one or more glycosylation markers, a peak ratio means a ratio between any one or more peaks and any other one or more peaks within the same glycosylation profile. In the method of determining a glycosylation marker, comparing peak ratios can mean comparing peaks intensities or comparing integrated areas under the peaks. In some embodiments of the method of determining glycosylation marker, comparing peak ratios can be carried for glycans of the tested and control samples which were not digested with one or more exoglycosidases. In some embodiments, comparing peak ratios can be carried out on the glycans which were digested with one or more exoglycosidases. In some embodiments, comparing peak ratios can be carried out for the glycans which were not digested with exoglycosidase and for the glycans digested with one or more exoglycosidases.

In some embodiments, measuring glycoprofiles with HPLC can be complemented with a mass spectrometry measurement. Complementary mass spectrometry data, such as MALDI, ESI or LC/MS) can serve, for example, for validation HPLC measured glycoprofiles as a separate orthogonal technique able to resolve the structures of more complex glycans when a sufficient amount of sample of a body fluid or a body tissue is available. Mass spectrometry used in combination with HPLC can be a powerful tool for structural analysis of glycoproteins. Mass spectrometry alone can be used for structural analysis of glycans providing monosaccharide composition of glycans. However, mass spectrometry used by itself does not distinguish isobaric monosaccharide (and hence oligosaccharides or glycans) and does not provide the information on monosaccharide linkage in glycans. The LC-MS/(MS) techniques can provide the most informative data out of the mass spectrometry technique, see Caesar, J. P., Jr., Sheeley, D. M. and Reinhold, V. N. (1990). “Femtomole oligosaccharide detection using a reducing-end derivative and chemical ionization mass spectrometry.” Anal Biochem 191: 247-52; Mattu, T. S., Pleass, R. J., Willis, A. C., Kilian, M., Wormald, M. R., Lellouch, A. C., Rudd, P. M., Woof, J. M. and Dwek, R. A. (1998). “The glycosylation and structure of human serum IgA1, Fab, and Fc regions and the role of N-glycosylation on Fc alpha receptor interactions.” Journal of Biological Chemistry 273: 2260-72; and Royle, L., Mattu, T. S., Hart, E., Langridge, J. I., Merry, A. H., Murphy, N., Harvey, D. J., Dwek, R. A. and Rudd, P. M. (2002). “An analytical and structural database provides a strategy for sequencing O-glycans from microgram quantities of glycoproteins.” Anal Biochem 304: 70-90. In some embodiments, measuring glycoprofiles by LC/MS can comprise using the LC stage of LC/MS not only for cleanup and preliminary separation of glycans before they enter the MS stage of LC/MS but for obtaining preliminary assignment of glycan structures in the glycans. This can be accomplished, for example, by using NP-HPLC matrix, for example NP-HPLC with TSK gel amide 80 matrix, in the LC column of LC/MS. In NP-HPLC with TSK gel amide 80 matrix, hydroxyl groups of glycans interact with the amide functionality, therefore, the elution order is determined by the number of hydroxyl groups in a particular glycan, its molecular confirmation and its relative solubility in the mobile phase.

In some embodiments, when the glycan pool comprises charged glycans, the glycan pool can be fractioned into several aliquots based upon charge. Fractioning of the glycan pool can be carried out, for example, by weak ion exchange (WAX) chromatography. Each WAX aliquot can be then analyzed independently by NP-HPLC combined with exoglycosidase digestions. Measuring glycoprofiles by WAX HPLC is described, for example, in Guile, G. R., Wong, S. Y. and Dwek, R. A. (1994). “Analytical and preparative separation of anionic oligosaccharides by weak anion-exchange high-performance liquid chromatography on an inert polymer column.” Analytical Biochemistry 222: 231-5.

Measuring glycoprofile of the glycans with the above described methods can allow detecting a particular glycan structure present in the glycans in subpicomole levels. Accordingly, in some of the embodiments, measuring glycoprofiles of the glycans is carried out using a technique able to detect a glycan structure present in the glycans in amount of 1 picomole, preferably 0.1 picomole, yet more preferably 0.01 picomole.

The methodology for diagnosing and monitoring an autoimmune disease can be illustrated in more details by the following example, however, it should be understood that the present invention is not limited thereto.

The invention is further illustrated by, though in no way limited to, the following examples.

### EXAMPLE

The measurement of the G0/triple-G1 ratio directly from undigested glycans released from whole serum was compared with the ‘classic’ measurement of the amount of G0 glycans as a percentage of the total glycans released from purified IgG after sialidase and fucosidase digestion. It has been shown that G0 released from purified IgG is disease(RA) specific marker that correlates with disease progression and that can be used as a prognostic indicator of the disease, see e.g. U.S. Pat. No. 4,659,659 “Diagnostic Method for Diseases Having an Arthritic Component” to Dwek et. al. issued on Apr. 21, 1987; Parekh et al., see “Association of Rheumatoid Arthritis and Primary Osteoarthritis with Changes in the Glycosylation Pattern of Total Serum IgG,” Nature, 316, pp. 452-457, 1985; and Parekh et. al. “Galactosylation of IgG Associated Oligosaccharides Is Reduced in Patients with Adult and Juvenile Onset Rheumatoid Arthritis and Is Related to Disease Activity”, Lancet, No. 8592, vol. 1, pp. 966-969, 1988. This study is used to demonstrate that a direct measurement of glycans released from whole serum can be used as marker for rheumatoid arthritis without IgG purification by correlating G0/triple-G1 ratio from undigested glycans released from whole serum with the amount of G0 glycans as a percentage of the total glycans released from purified IgG.

Selection of patient sample. Control patient serum was pooled discarded clinical material from individuals undergoing routine employee health screening. RA patients were selected based on a combination of physician global activity score, rheumatoid factor seropositivity and active joint count.

IgG purification: IgG was isolated from whole serum via affinity chromatography employing protein-G sepharose as described in “Antibodies: A laboratory manual”, Cold Spring Harbor Laboratory, Cold Spring Harbor, 1988, and P. L. Ey et. al. “Isolation of pure IgG₁, IgG₂ₐ and IgG_{2b} immunoglobulins from mouse serum using protein A-Sepharose”, Molecular Immunology, vol. 15, pp. 429, 1978, both incorporated herein by reference in their entirety. Briefly, 100 μl of whole serum was diluted with 300 μl of 100 mM Tris pH 8.0 and allowed to pass over a 1 ml column of protein-G sepharose beads (Amersham Biosciences). Bound material was washed with 15 column volumes of 100 mM Tris pH 8.0. IgG was eluted using 100 mM glycine pH 2.6 buffer directly into 1/10 volume 1M Tris pH 8.0 and collected in 1 ml fractions. Protein content of eluted fractions was determined by 280 nM (UV) absorbance (Beckman Coulter Model DU640 spectrophotometer). Protein containing eluted fractions were pooled and dialyzed into phosphate buffered saline. IgG presence in eluted fractions was confirmed via 10% polyacryl amide gel electrophoresis (PAGE) under reducing conditions (as described, e.g., in Laemmli, “Cleavage of structural proteins during the assembly of the head of bacteriophage T4”, Nature,: 227, 680-685, 1970, incorporated herein by reference in its entirety) and via western blot (Current Protocols in Immunology. John Wiley and Sons, 1994, incorporated herein by reference in its entirety) utilizing horseradish-peroxidase conjugated donkey-anti-human IgG (Jackson Immunochemicals) and visualized with Western Lightning Chemiluminescence Reagent Plus (Perkin Elmer). Quantitative depletion of serum IgG in column flow through material was confirmed via western blot analysis.

Glycans release: Glycans were released from purified IgG by running the reduced and alkylated sample on sodium-dodecyl sulphate polyacryl amide gel electrophoresis (SDS-PAGE), cutting out the heavy chain and digesting with peptide N-glycosidase F (PNGaseF) as described in Küster, B., Wheeler, S. F., Hunter, A. P., Dwek, R. A., and Harvey, D. J (1997). Sequencing of N-linked oligosaccharides directly from protein gels: in-gel deglycosylation followed by matrix-assisted laser desorption/ionization mass spectrometry and normal-phase high-performance liquid chromatography. Analytical Biochemistry 250: 82-101, incorporated herein by reference in its entirety. Glycans were released with PNGaseF from 5 μl of whole sera after binding the reduced and alkylated serum to MultiScreen_IP, 0.45 μm hydrophobic, high protein binding polyvinylidene fluoride (PVDF) membranes in a 96 well plate format (Millipore, Bedford, Mass., USA). Released glycans were labeled with 2AB fluorescent label (Ludger Ltd, Oxford, UK) as described in Bigge, J. C., Patel, T. P., Bruce, J. A., Goulding, P. N., Charles, S. M., and Parekh, R. B. (1995). Nonselective and efficient fluorescent labeling of glycans using 2-amino benzamide and anthranilic acid. Analytical Biochemistry 230: 229-238, incorporated herein by reference in its entirety, and run by normal phase high performance liquid chromatography (NP-HPLC) on a 4.6×250 mm TSK Amide-80 column (Anachem, Luton, UK) using a Waters 2695 separations module equipped with a Waters 2475 fluorescence detector (Waters, Milford, Mass., USA) as described in Guile, G. R., Rudd, P. M, Wing, D. R., Prime, S. B., and Dwek, R. A. (1996). A rapid high-resolution high-performance liquid chromatographic method for separating glycan mixtures and analyzing oligosaccharide profiles. Analytical Biochemistry 240: 210-226. Purified, 2AB labeled IgG heavy chain glycans were also digested with sialidase and fucosidase to reduce all the structures to G0, G1 or G2±bisect, then run on NP-HPLC. [G0 denotes no galactose; G1, one galactose; and, G2 two galactose, all on biantennary complex N-glycans.]

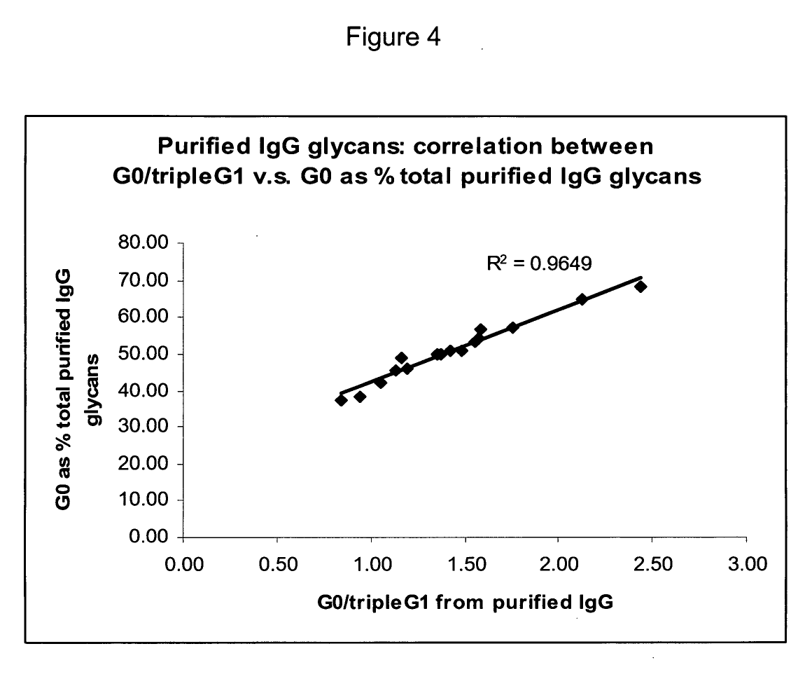

Statistical analysis. All the data for glycan ratios are listed in Table 1. FIGS. 4, 5 and 6 are plots showing correlations between these data. The R² values were obtained by linear regression analysis using Microsoft Excel.

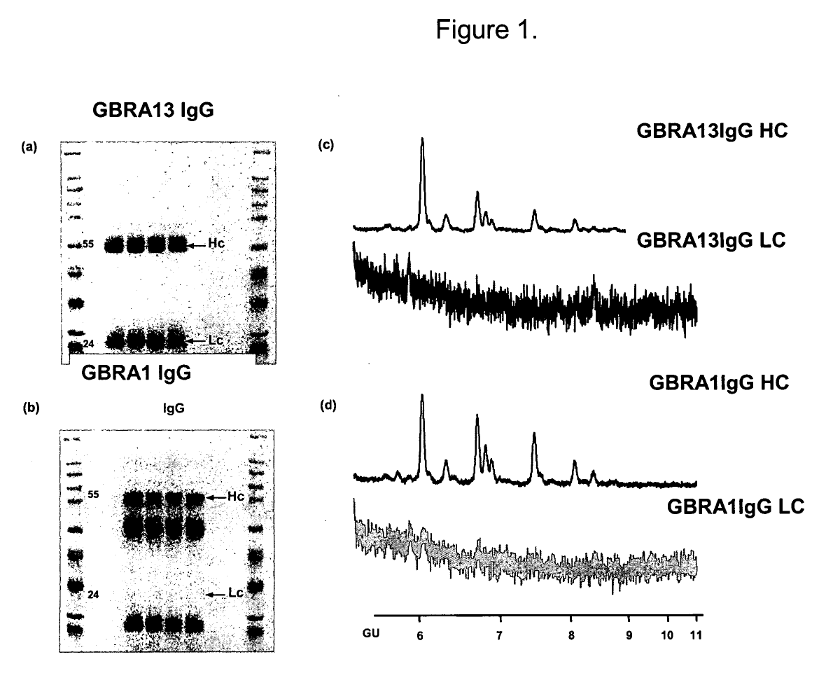

Experimental results. FIG. 1 shows SDS-PAGE and NP-HPLC profiles from samples GBRA1 and GBRA13. In particular, insets (a) and (b) of FIG. 1 provide SDS-PAGE gel pictures of the purified IgGs from the respective samples separated into heavy (H) and light (L) chain bands. Insets (c) and (d) of FIG. 1 provide NP-HPLC profiles for heavy and light chain glycans released from the gel bands shown in (a) and (b) and not subjected to digestion with sialidase and fucosidase. Since no glycans were detected on the light chain, only the heavy chain was required for analysis.

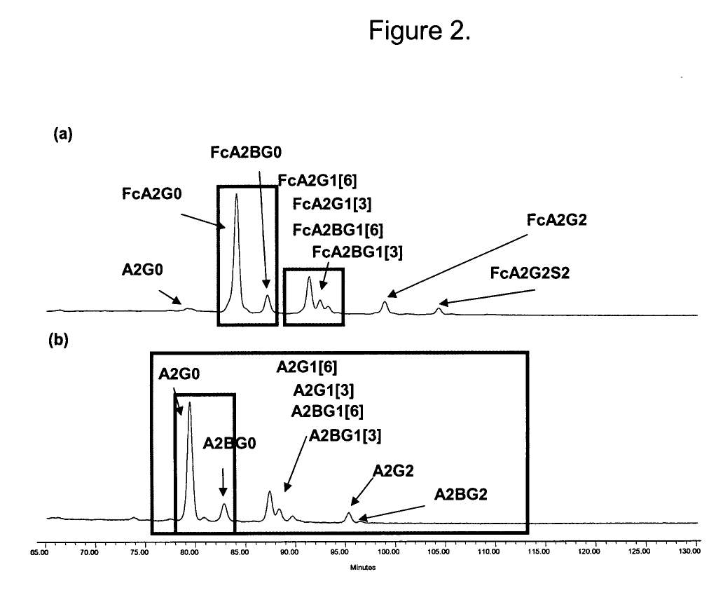

FIG. 2 illustrates the details of (a) the measurement of the G0/triple-G1 ratio directly from undigested glycans released from purified IgG and (b) the ‘classic’ measurement of the ratio G0 glycans to the total glycans released from purified IgG and digested with sialidase and fucosidase. In particular, FIG. 2 shows NP-HPLC profiles from the sample GBRA15. Each peak corresponds to certain glycan(s). The peaks in each profile are integrated to give the area under the curve for each peak. In the measurement of the G0/triple-G1 ratio, the area under the peaks corresponding to the G0 glycans (left box of the inset (a) of FIG. 2) are divided by the area under the triplet of peaks corresponding to the G1 glycans (right box of the inset (a) of FIG. 2). As the vast majority of glycans found in these experiments were core fucosylated, only core fucosylated glycans were included in these measurements, i.e. the ratio G0/triple-G1 is actually the peak area of FcA2G0 divided by the peak area of FcA2G1[6]+FcA2G1[3]+FcA2BG1[6]+FcA2BG1[3](which elutes as a triplet).

In the ‘classic’ measurement, the area under the peaks corresponding to the G0 peaks is divided by the total area under all the peaks in the profile and expressed as a percentage.

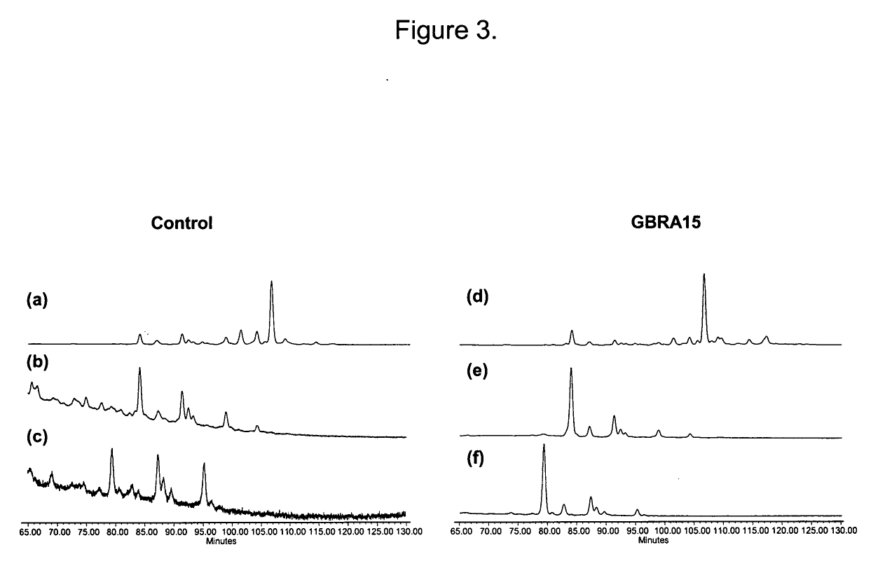

FIG. 3 illustrates NP-HPLC profiles of control sample and the sample GBRA15.

Particularly, insets (a) and (d) show glycans released from whole sera of the respective samples, insets (b) and (e) show undigested heavy chain glycans released from respective purified IgGs, insets (c) and (f) show heavy chain glycans released from respective purified IgGs and digested with sialidase and fucosidase.

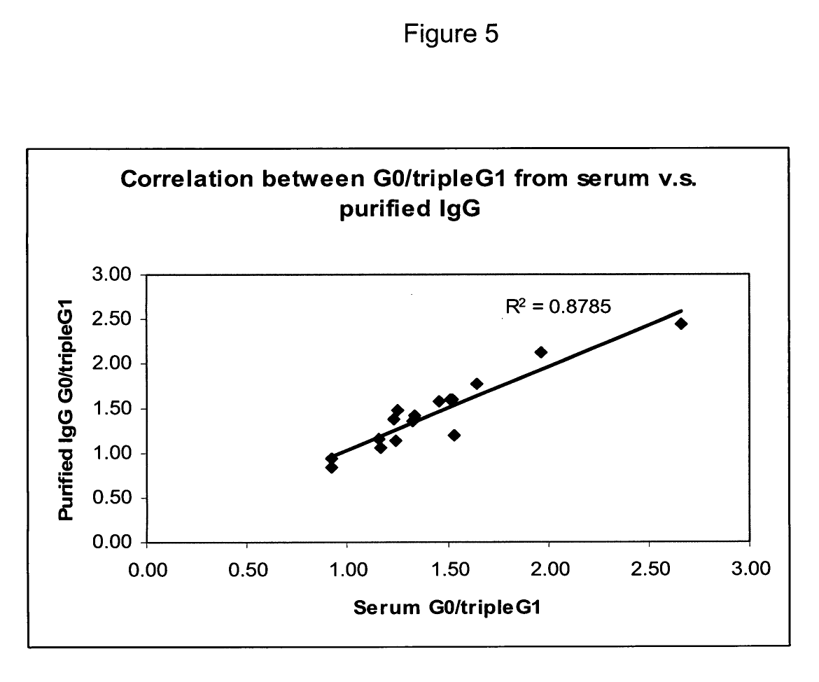

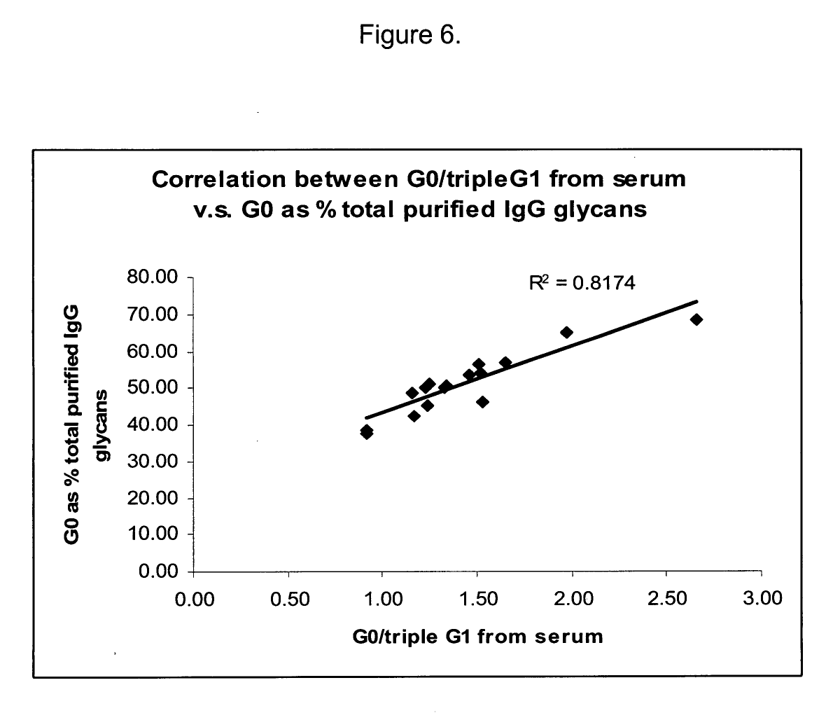

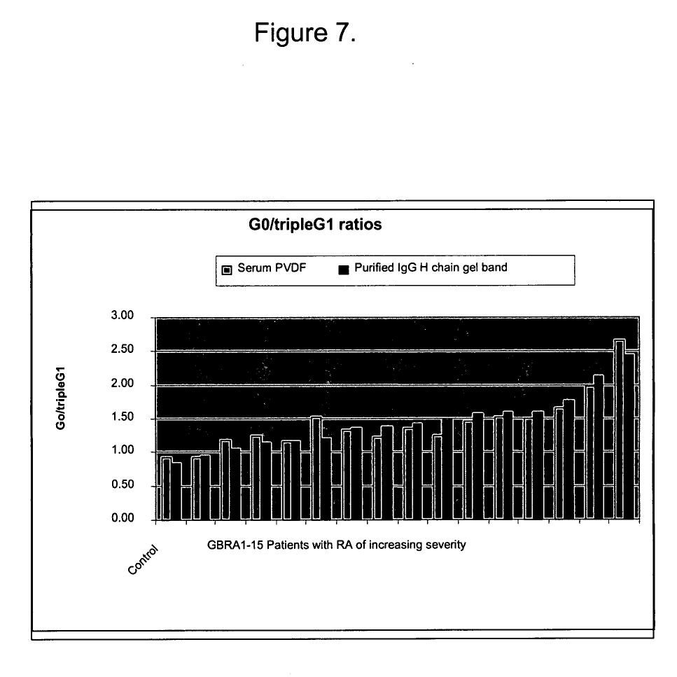

Table 1 lists the ratios of the G0 to triple-G1 peak from whole serum and purified IgG from the same serum samples from 15 RA patients and one pooled control. The ‘classic’ measurement of the amount of G0 glycans as a percentage of the total glycans (G0+G1+G2) from purified IgG is also shown. Comparing the results of the two different measurements taken from purified IgG, a high correlation (R²=0.9649) is found, indicating that the ratio G0/triple-G1 is as a good measurement as the ‘classic’ measurement of the percentage of G0 glycans in total glycan pool (FIG. 4). Comparing the G0/triple-G1 ratio between purified IgG and whole serum glycans gives a correlation of R²=0.8785 (FIG. 5), whilst comparing the G0/triple-G1 ratio from whole serum glycans with the percentage G0 glycans from purified IgG gives a correlation of R²=0.8174 (FIG. 6). FIG. 7 is a histogram showing the G0/triple-G1 ratios for all serum and IgG samples.

TABLE 1

| | | | G0 as % of TOTAL |
|---|---|---|---|
| | undigested | | digested IgG |
| | G0/triple-G1 | | glycans |
| | Glycans released from | Glycans released from | |
| Patient i.d. | Serum using PVDF | purified IgG using SDS-PAGE | |
| Control | 0.92 | 0.84 | 37.40 |
| GBRA1 | 0.92 | 0.94 | 38.43 |
| GBRA2 | 1.17 | 1.05 | 42.26 |
| GBRA3 | 1.24 | 1.13 | 45.43 |
| GBRA4 | 1.16 | 1.16 | 48.74 |
| GBRA5 | 1.53 | 1.19 | 46.26 |
| GBRA6 | 1.33 | 1.35 | 49.94 |
| GBRA7 | 1.23 | 1.37 | 50.18 |
| GBRA8 | 1.34 | 1.42 | 50.74 |
| GBRA9 | 1.25 | 1.48 | 51.14 |
| GBRA10 | 1.46 | 1.56 | 53.50 |
| GBRA11 | 1.52 | 1.58 | 54.13 |
| GBRA12 | 1.51 | 1.59 | 56.59 |
| GBRA13 | 1.65 | 1.76 | 56.98 |
| GBRA14 | 1.97 | 2.13 | 65.16 |
| GBRA15 | 2.66 | 2.44 | 68.28 |

Conclusion. The use of the high throughput PVDF membrane 96 well plate format with only 5 μl of whole serum being used to obtain glycans for a direct measurement of the G0/triple-G1 ratio has been demonstrated. This procedure replaces the more lengthy procedure of measuring the percentage of G0 glycans in the glycans released from purified IgG determined after exoglycosidase treatment, as an indicator of RA disease state. Thus, to monitor the RA disease state, one can efficiently reduce working hours from sample preparation to results by using the PVDF membrane method with whole serum as well as reducing the amount of material (serum) used.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention.

All of the publications, patent applications and patents cited in this specification are incorporated herein by reference in their entirety.

### Additional Embodiments

1. A method for diagnosing and monitoring an autoimmune disease comprising

releasing glycans of glycoproteins from samples of a body fluid without purifying the glycoproteins, and without exposing the body fluid to hydrazinolysis;

quantitatively analyzing the glycans.

2. The method of embodiment 1, wherein the body fluid is a whole serum, a blood plasma, a synovial fluid, urine, seminal fluid, or saliva.

3. The method of embodiment 1, wherein the body fluid is a whole serum.

4. The method of embodiment 1, wherein releasing glycans comprises preparing a gel from the body fluid.

5. The method of embodiment 4, wherein the glycans are N-glycans and releasing glycans further comprises releasing the N-glycans from the gel using PNGaseF enzyme.

6. The method of embodiment 1, wherein releasing glycans comprises attaching glycoproteins to polyvinyldene fluoride membranes.

7. The method of embodiment 6, wherein the glycans are N-glycans and releasing glycans further comprises incubating polyvinyldene fluoride membranes with PNGaseF enzyme.

8. The method of embodiment 6, wherein releasing glycans further comprises chemically releasing the glycans by β-elimination.

9. The method of embodiment 6, wherein releasing glycans further comprises releasing the glycans by ammonia-based β-elimination.

10. The method of embodiment 1, further comprising labeling the glycans before quantitatively analyzing the glycans with a radioactive label or a fluorescent label.

11. The method of embodiment 10, wherein the fluorescent label is 2-aminobenzamide.

12. The method of embodiment 1, wherein quantitatively analyzing the glycans comprises analyzing the glycans by chromatography, mass spectrometry or a combination thereof.

13. The method of embodiment 12, wherein the chromatography is high performance liquid chromatography.

14. The method of embodiment 12, wherein quantitatively analyzing the glycans further comprises obtaining glycosylation profiles of the glycans, wherein each of the glycosylation profiles corresponds to one of the samples and wherein each of the glycosylation profiles comprises a plurality of peaks.

15. The method of embodiment 14, wherein the samples comprise diseased samples and one or more control samples, wherein diseased samples are body fluid samples of autoimmune disease patients and control samples are body fluid samples of patients without the autoimmune disease, and wherein the glycosylation profiles comprise diseased glycosylation profiles corresponding to the diseased samples and one or more control glycosylation profiles corresponding to the one or more control samples.

16. The method of embodiment 15, wherein quantitatively analyzing the glycans comprises comparing peak ratios in the diseased glycosylation profiles and in the one or more control glycosylation profiles and selecting out of the peak ratios a glycosylation marker of the autoimmune disease, wherein the glycosylation marker is a ratio having a highest correlation with parameters of the autoimmune disease patients out of the peak ratios.

17. The method of embodiment 16, wherein the parameters of the autoimmune disease patients are diagnosis, age, sex, disease activity, disease prognosis, remission, response to a therapy or a combination thereof.

18. The method of embodiment 16, further comprising applying the glycosylation marker to diagnosing the autoimmune disease, monitoring the autoimmune disease, prognosticating the autoimmune disease, or predicting response to a therapy in one or more new patients.

19. The method of embodiment 1, wherein the autoimmune disease is rheumatoid arthritis, osteoarthritis, juvenile chronic arthritis, systematic lupus erythematosus, Sjogren's syndrome, ankylosing spondylitis, psoriatic arthritis, multiple sclerosis, inflammatory bowel disease, graft-vs-host disease or scleroderma.

20. The method of embodiment 1, wherein the autoimmune disease is rheumatoid arthritis.

21. A method of diagnosing and monitoring an autoimmune disease comprising

measuring diseased glycosylation profiles and one or more control glycosylation profiles, wherein the diseased glycosylation profiles are glycosylation profiles of glycans of glycoproteins from autoimmune disease patients and the one or more control glycosylation profiles are glycosylation profiles of glycans of glycoproteins from patients without the autoimmune disease and wherein measuring diseased glycosylation profiles and one or more control glycosylation profiles is carried out by HPLC or a combination of HPLC and mass spectrometry;

comparing peak ratios in the diseased glycosylation profiles and in the one or more control glycosylation profiles and selecting a ratio having a highest correlation with parameters of the autoimmune disease patients out of the peak ratios as a glycosylation marker of the autoimmune disease.

22. The method of embodiment 21, wherein the parameters of the autoimmune disease patients are diagnosis, age, sex, disease activity, disease prognosis, remission, response to a therapy or a combination thereof.

23. The method of embodiment 21, further comprising applying the glycosylation marker to diagnosing the autoimmune disease, monitoring the autoimmune disease, prognosticating the autoimmune disease, or predicting a response to a therapy in one or more new patients.

24. The method of embodiment 21, wherein the autoimmune disease is rheumatoid arthritis, osteoarthritis, juvenile chronic arthritis, systematic lupus erythematosus, Sjogren's syndrome, ankylosing spondylitis, psoriatic arthritis, multiple sclerosis, inflammatory bowel disease, graft-vs-host disease or scleroderma.

25. The method of embodiment 22, wherein the glycans are released without purifying the glycoproteins.

26. The method of embodiment 22, wherein the glycans are released from purified glycoproteins.

27. The method of embodiment 22, wherein the glycans are released from purified serum IgG.

28. A high throughput method for diagnosing and monitoring rheumatoid arthritis in a patient comprising

releasing glycans of glycoproteins from a body fluid or a body tissue of the patient;

measuring a ratio between an amount of G0 glycans and an amount of G1 glycans in the glycans.

29. The method of embodiment 28, wherein the body fluid is a whole serum, a blood plasma or a synovial fluid.

30. The method of embodiment 28, wherein measuring a ratio is carried out by chromatography, mass spectrometry or a combination thereof.

31. The method of embodiment 28, wherein releasing glycans does not comprise purifying the glycoproteins.

32. The method of embodiment 28, wherein releasing glycans does not comprise treating the glycans with exoglycosidase.

33. The method of embodiment 28, wherein releasing glycans does not comprise exposing the body fluid or the body tissue to hydrazinolysis.

34. The method of embodiment 28, wherein the glycoproteins are purified glycoproteins.

What is claimed is:

1. A method of identifying and/or quantifying one or more glycosylation markers of an autoimmune disease, comprising
(A) obtaining a sample from a subject diagnosed with the autoimmune disease;
(B) releasing glycans of unpurified glycoproteins of the sample;
(C) measuring a glycosylation profile of the glycans by quantitative high performance liquid chromatography alone or in combination with mass spectrometry; and
(D) comparing the glycosylation profile with a control profile to determine the one or more glycosylation markers of the autoimmune disease.

2. The method of claim 1, wherein the subject is a human.

3. The method of claim 1, wherein the sample is a sample of a body fluid of the subject.

4. The method of claim 3, wherein the body fluid is whole serum, blood plasma, a synovial fluid, urine, seminal fluid, or saliva.

5. The method of claim 4, wherein the body fluid is whole serum.

6. The method of claim 1, wherein the autoimmune disease is rheumatoid arthritis, osteoarthritis, juvenile chronic arthritis, systematic lupus erythematosus, Sjogren's syndrome, ankylosing spondylitis, psoriatic arthritis, multiple sclerosis, inflammatory bowel disease, graft-vs-host disease or scleroderma.

7. The method of claim 6, wherein the autoimmune disease is rheumatoid arthritis.

8. The method of claim 1, wherein said unpurified glycoproteins are total glycoproteins of the sample.

9. The method of claim 1, wherein said unpurified glycoproteins are a selection of total glycoproteins of the sample.

10. The method of claim 1, further comprising immobilizing said unpurified glycoproteins prior to said releasing.

11. The method of claim 10, wherein said immobilizing is immobilizing in a high through put format.

12. The method of claim 10, wherein said immobilizing is immobilizing on a protein binding membrane.

13. The method of claim 10, wherein said immobilizing in a gel piece or a gel block.

14. The method of claim 1, wherein the glycans are N-linked glycans or O-linked glycans.

15. The method of claim 1, wherein said comparing comprises comparing one or more peak ratios in the measured glycosylation profile and the control profile.

16. The method of claim 1, further comprising selecting a best glycosylation marker out of the one or more glycosylation markers, wherein the best glycosylation marker has a highest correlation with one or more parameters of the subject.

17. The method of claim 16, wherein said parameters are diagnosis, age, sex, disease activity, disease prognosis, remission, response to a therapy or a combination thereof.

18. The method of claim 1, further comprising labeling the released glycans with a fluorescent label prior to the measuring.

19. The method of claim 18, wherein the fluorescent label is 2-aminobenzamide.

20. A method of diagnosing, monitoring and/or prognosticating an autoimmune disease in a subject, comprising
(A) obtaining a sample from the subject;
(B) releasing glycans of unpurified glycoproteins of the sample;
(C) measuring a glycosylation profile of the glycans by quantitative high performance liquid chromatography alone or in combination with mass spectrometry; and
(D) comparing the glycosylation profile with a control profile to determine a level of one or more glycosylation markers of the autoimmune disease.

21. The method of claim 20, wherein the autoimmune disease is rheumatoid arthritis, osteoarthritis, juvenile chronic arthritis, systematic lupus erythematosus, Sjogren's syndrome, ankylosing spondylitis, psoriatic arthritis, multiple sclerosis, inflammatory bowel disease, graft-vs-host disease or scleroderma.

22. The method of claim 21, wherein the autoimmune disease in rheumatoid arthritis.

23. The method of claim 22, wherein the glycosylation marker is a ratio between an amount of G0 glycans and an amount of G1 glycans.

24. A method of diagnosing, monitoring and/or prognosticating rheumatoid arthritis in subject, comprising
(A) obtaining a sample of the subject;
(B) releasing glycans of glycoproteins of the sample; and
(C) measuring a glycoprofile of the glycans to determine a ratio between an amount of G0 glycans and an amount of G1 glycans.

25. The method of claim 24, wherein said measuring is measuring by quantitative high performance liquid chromatography.

26. The method of claim 24, wherein said glycoproteins are unpurified glycoproteins of the sample.

27. The method of claim 26, wherein the unpurified glycoproteins are total glycoproteins of the sample.

28. The method of claim 26, wherein the unpurified glycoproteins are a selection of total glycoproteins of the sample.

29. The method of claim 24, wherein the glycoproteins are purified glycoproteins of the sample.

30. The method of claim 24, further comprising immobilizing the glycoproteins prior to the releasing.

31. The method of claim 30, wherein the immobilizing is immobilizing in a high throughput format.
